(19) United States
(12) Patent Application Publication
(10) Pub. No.: US 2026/0317701 A1
(43) Pub. Date: Oct. 15, 2026

(54) METHOD FOR CULTURING ANIMAL CELLS OR ANIMAL CELL LINE

(71) Applicant: CELLMEAT CORP., Seoul (KR)

(72) Inventors: Gil Jun PARK, Gwangju (KR); Hee Jung KIM, Gwangju (KR); Min Soo LEE, Gwangju (KR); Ji Min CHA, Hwasun-gu (KR); Ha Yeon CHOI, Gwangju (KR); Soo Yong PARK, Gwangju (KR); Su Bin HA, Gwangju (KR); Seul Gi LEE, Gwangju (KR)

(21) Appl. No.: 19/104,522

(22) PCT Filed: Aug. 18, 2023
(86) PCT No.: PCT/KR2023/012255

§ 371 Date: Feb. 18, 2025

(30) Foreign Application Priority Data
Aug. 19, 2022 (KR) 10-2022-0104288
Aug. 19, 2022 (KR) 10-2022-0104306
Sep. 23, 2022 (KR) 10-2022-0121025
Sep. 23, 2022 (KR) 10-2022-0121026
Feb. 16, 2023 (KR) 10-2023-0020437

(51) Int. Cl. C12N 5/07; A23L 13/00

(52) U.S. Cl. CPC C12N 5/0601; A23L 13/00; C12N 2500/32; C12N 2500/74

(57) ABSTRACT

The present invention relates to: an animal cell culture medium composition; a method for culturing animal cells using same; a method for preparing a non-human animal cell line; a cell line prepared using same; and a method for culturing the cell line. An animal cell culture medium composition containing yeast extract or peptone according to an aspect has the effects of allowing the growth and proliferation of animal cells even in a medium not containing serum, and promoting the growth and proliferation of animal cells. An animal cell culture medium composition containing a basic medium and yeast extract according to an aspect has the effects of allowing the effective proliferation of cells even in a basic medium not containing vitamins and organic acids, and reducing the price of a cell culture medium by minimizing culture medium components. A method for preparing a cell line according to an aspect, the method including a step for separating cells from non-human animals and a step for suspension-culturing the separated cells, has the effect of making it possible to effectively obtain a cell line from non-human animals.

### TECHNOLOGY

The present invention relates to a medium composition for animal cell culture, a method of culturing animal cells using the same, a method of preparing a non-human animal cell line, a cell line prepared thereby, and a method of culturing cell line.

### BACKGROUND

With the continuous increase in population, the problem of environmental pollution is emerging, and in particular, the problem of marine environmental pollution is intensifying to a serious level. Since the 1960s, environmental pollution and indiscriminate fishing have pushed some species to unsustainable levels. Marine life may suffocate due to trash, but the more serious problem is that plastic garbage floating in the sea is directly exposed to ultraviolet rays and produces microplastics. These microplastics gradually accumulate in the bodies of marine organisms and cause various diseases in the body, threatening the survival of marine organisms. Furthermore, the accumulation of microplastics in humans who consume marine organisms with accumulated microplastics is also taken seriously. In addition, rapid climate change due to global warming is gradually increasing the temperature of the ocean, further fueling the decline of marine species.

Despite the destruction of marine ecosystems, the demand for marine resources is increasing due to the increase in population, and the demand for crustaceans such as shrimp, crabs, and lobsters continues to increase. As a result of the increasing demand, aquaculture has been expanding, but this has also led to various side effects. However, the increase in aquaculture has intensified the emission of carbon gases, which are the main substances of global warming, and the supply method to meet demand is causing new environmental pollution. In addition, while creating aquaculture spaces, it destroys ecological resources such as coastal ecosystems, which function to absorb carbon, one of the causes of global warming. Moreover, there is a risk that mass inhabitation in a dense space may lead to pollution due to increased waste in nearby waters and mass mortality due to viral infection.

Cultured meat has been proposed as a solution to curb environmental pollution and meet demand. Cultured meat can reduce the emission of carbon gases because it involves extracting cells from a single individual and multiplying them to create multiple individuals, and it also shows a high production yield per unit area. In addition, since cultured meat multiplies cells in the incubator of the laboratory, it has the advantage of providing safe food that is not contaminated with heavy metals and microplastics, unlike seafood caught in the sea, and it has the advantage of being sustainable and does not destroy the marine environment.

In order to develop and produce cultured meat, cell extraction technology, cell proliferation technology, and cell-specific culture media must be combined, and the biggest problem for commercialization is to reduce the cost of culture media. Fetal bovine serum (FBS), which typically contains proteins, amino acids, hormones, etc.), is added at a rate of 5 to 20% to culture cells and the FBS is considered to be essential in cell culture and very effective for cell growth and proliferation, but there are significant disadvantageous as it is directly extracted from the blood of a cow fetus, which is highly unethical and quite expensive.

In addition, the most important thing for commercializing cultured meat is the reduction of the price of cultured meat production, and the biggest factor that can influence on the price reduction for the production of cultured meat is the cell culture medium. The basic culture medium used in the cultivation of cultured meat contains a variety of ingredients. If cultured meat can be produced using cultured media without these ingredients, it can greatly help reduce the cost.

In addition, the main technique for producing crustacean cultured meat is to establish a technique to isolate and cultivate cells from the tissues of cultured meat individuals to be made. While the method of isolating and cultivating muscle cells from shrimp has been reported earlier (George et al., In Vitro Cell. Dev. Biol.—Animal 2010), it has been shown that the cells stop growing after culture for a certain period of time (<7 days) due to the limitation of proliferation capacity.

Accordingly, the present inventors have completed the present invention by developing a culture medium in which the growth and proliferation of crustacean cells is actively carried out without serum or various ingredients, a cell separation method that can be commonly applied to crustaceans, and an effective cell culture method without limitation of proliferation capacity.

### DETAILED DESCRIPTION OF THE INVENTION

### Technical Challenges

One aspect is to provide a medium composition for animal cell culture, comprising yeast extract or peptone.

Another aspect is to provide a medium composition for animal cell culture, comprising a basic medium and yeast extract.

Another aspect is to provide a method of culturing animal cells, comprising culturing cells in a medium composition for animal cell culture comprising yeast extract or peptone.

Another aspect is to provide a method of culturing animal cells, comprising culturing cells in a medium composition for animal cell culture, comprising a basic medium and yeast extract.

Another aspect is to provide a method of preparing a cell line, comprising:
separating a cell from a non-human animal; and
suspension-culturing the separated cell.

Another aspect is to provide a cell line prepared according to the aforesaid preparing method.

Another aspect is to provide a method of culturing a cell line, comprising performing suspension culture for proliferation of a cell line.

### Means of Solving the Challenge

One aspect is to provide a medium composition for culturing animal cells, comprising yeast extract or peptone.

Another aspect is to provide a medium composition for culturing animal cells, comprising a basic medium and yeast extract.

Another aspect is to provide a method of culturing animal cells, comprising culturing cells in a medium composition for culturing animal cells, comprising yeast extract or peptone.

Another aspect is to provide a method of culturing animal cells, comprising culturing cells in a medium composition for culturing animal cells comprising a basic medium and yeast extract.

In one embodiment, the medium composition may include the yeast extract or peptones at 0.01 to 20, 0.01 to 10, 0.6 to 10, or 0.6 to 2% (w/v).

Peptone is a general term for the products produced when various proteins are enzymatically decomposed or hydrolyzed, and contains components such as polypeptides, amino acids, and minerals, which are mixed together.

In one embodiment, the peptones may be animal peptones, bacterial peptones, or vegetable peptones. They may be preferably vegetable peptones, but not limited thereto.

The animal peptones may be meat and/or casein.

The bacterial peptone may be Bacto Peptone.

In one embodiment, the vegetable peptones may be one or more species selected from a group consisting of a soybean peptone, wheat peptone, broadbean peptone, potato peptone, pea peptone, papaic soy peptone, and lupin peptone. They may be preferably soybean peptone, but not limited thereto.

In this specification, the term “vegetable peptone” refers to the breakdown product of proteins obtained from vegetables. For example, a soy peptone is a product obtained by breaking down the total protein obtained from soybeans. The decomposition of the vegetable protein can be done by partial digest. The decomposition of the protein can be done through decomposition by acid treatment, base treatment, enzymatic treatment, high pressure treatment, heat treatment or physical treatment. The physical treatment is, for example, grinding.

In the present invention, a vegetable peptone is a partial decomposition product of a vegetable protein, which may be in the form of a mixture containing both a peptide consisting of several or dozens of amino acids and a complete protein molecule, as well as a single molecule of amino acids.

The vegetable source from which the vegetable peptone of the present invention is obtained is not particularly limited, and includes any peptone derived from any vegetable if it has the ability to promote cell proliferation.

The medium composition of the present invention may include microbial extracts, and the microorganisms may be edible.

The microorganisms may be, but are not limited to, bacteria, archaea, or yeast.

In this specification, the term “yeast extract” refers to a natural biological material that is produced through self-digestion by NaCl, ethanol, or enzymatic treatment after yeast is cultured, so it does not cause problems in the production of harmful substances such as acid-degraded hydrolyzed vegetable protein (HVP), and is used in microbial fermentation media or natural seasonings.

The yeast extract may be made by hydrolysis of the polypeptides of edible yeast by adding edible enzymes to the edible yeast, and may be used for edible purpose.

In one embodiment, the yeast extract may be available for food.

Yeast extract is known to contain minerals, vitamins, nucleic acids, peptides, and amino acids.

In one embodiment, the yeast may be one or more species selected from a group consisting of Saccharomyces sp., Wicherhamomyces sp., Pichia sp. and Hanseniaspora sp.

The vegetable peptone and the yeast extract are vegetables or microbial-derived components, which may be substitutes for animal components. The vegetable or microbial-derived components are very suitable for use in biopharmaceuticals and food applications.

In one embodiment, the animal cell may be one or more species selected from a group consisting of muscle stem cells, muscle cells, and progenitors thereof.

The muscle stem cells may be satellite cells or myoblasts, for example, but not limited to myoblasts.

In this specification, the term “muscle stem cell” refers to a cell that has the characteristics of muscle stem cells, including all capabilities of proliferation without transformation, infinite proliferation, self-reproduction, and differentiation into muscle, and may be included without limitation if it exhibits the capabilities of self-reproduction or infinite proliferation, and differentiation into muscle. The capabilities of self-reproduction and differentiation into muscles can be identified as markers. Also, the muscle stem cell may be a cell exhibiting expression of CD29, CD56, Oct4, Nanog, or Pax7, along with, for instance, self-reproduction. If the muscle stem cell has differentiation and self-reproductive ability, their type and origin are not restricted. The muscle stem cell may be derived from, for example, mammals, humans, monkeys, pigs, horses, cows, chickens, ducks, sheep, dogs, cats, mice or rabbits. In one embodiment, the muscle stem cell may be a bovine or chicken muscle stem cell.

The muscle stem cells may be cells that exhibit the expression of Pax7 or MyoD in the process of being induced into myoblasts.

In this specification, the term “myoblast” refers to a precursor cell in the process of differentiation into muscle cells, which proliferate like a myocyte and can be differentiated into myogenic cells (myocytes) by fusion between cells.

In this specification, the term “muscle cells” refers to cells that can construct muscle tissue produced from myoblasts through the differentiation process of myoblasts.

The muscle cells may be, but are not limited to, myocytes or myotubes, e.g., myocytes.

The progenitor cells refer to cells capable of producing cells differentiated into multiple lineages, e.g., myoblasts, fibroblasts, adipocytes, stromal cells, epithelial cells, smooth muscle cells, and endothelial cells. Progenitor cells differ from stem cells in that they typically do not have the ability to self-replicate extensively.

The animal cells may include, but are not limited to, fat cells (fat cells or adipocytes) and/or progenitor cells thereof, stromal cells (connective tissue) and/or progenitor cells thereof, or endothelial cells (blood vessels) and/or progenitor cells thereof.

In one embodiment, the animal may be, but is not limited to, one or more species selected from a group consisting of cattle, sheep, pigs, poultry, crustaceans and fish.

In one embodiment, the animal cell may refer to an animal-derived cell produced by isolating cells from an animal's tissue and culturing them in vitro.

In one embodiment, the animal cell may be a crustacean cell.

In one embodiment, the animal cell may be a shrimp, crab, or lobster-derived cell.

In this specification, the term “culture media” refers to substances that can support the growth and survival of cells, including stem cells, in vitro.

In this specification, the term “basic medium” means a medium containing the basic components for culturing cells. In general, when cells are cultured using only a basic medium, the cells often do not proliferate smoothly, so various ingredients are added for optimal cell proliferation.

In one embodiment, the culture media or basic medium may be, but are not limited to, one or more species selected from a group consisting of DMEM (Dulbecco's Modified Eagle's Medium), MEM (Minimal Essential Medium), BME (Basic medium Eagle), RPMI 1640, DMEM/F-10 (Dulbecco's Modified Eagle's Medium: Nutrient Mixture F-10), DMEM/F-12 (Dulbecco's Modified Eagle's Medium: Nutrient Mixture F-12), a-MEM (a-Minimal Essential Medium), G-MEM (Glasgow's Minimal Essential Medium), IMDM (Isocove's Modified Dulbecco's Medium), Knockout DMEM, E8 (Essential 8 Medium), SF-DMEM (serum free-Dulbecco Modified Eagle Medium), L15 Medium, and Grace's Insect Medium.

In addition to the aforementioned culture media, the media used in the industry are sufficient, and may contain amino acids, vitamins, mineral salts, and other ingredients. For example, non-essential amino acids and L-glutamine, vitamins such as vitamin B12, inorganic salts such as trace components (CuSO45H20, Fe(NO339H20, ZnSO4), phosphoenol pyruvate, monoethanolamine, sodium selenite and sodium bicarbonate, and other ingredients such as d-glucose, linoleic acid. Linoleic acid, lipoic acid, sodium pyruvate, hypoxantine Na, putrescine HCl, and polyamine solutions may be included.

In one embodiment, the culture media may be a serum-free medium.

In one embodiment, the medium composition for animal cell culture may not contain serum.

A serum-free medium refers to a cell culture medium that does not use serum, which has ethical, environmental, and high-cost issues, and eliminates the use of serum.

In one embodiment, the culture media may be a medium containing serum.

The serum may be, but is not limited to, animal blood, preferably derived from the blood of mammals (e.g., pigs, horses, cattle, goats, sheep and dogs), and may preferably be FBS.

In one embodiment, the serum may be, but is not limited to, one or more species selected from a group consisting of FBS, human platelet lysate (hPL), human serum albumin (HSA), human serum (HS), platelet-rich plasma (PRP), platelet poor plasma (PPP), calf serum, horse serum, porcine serum, and sheep serum.

In one embodiment, the culture may be a floating culture.

In this specification, the term “cell culture” refers to the process of artificially growing living cells in vitro under controlled conditions. It may also be aseptically removing a portion the individual tissue, decomposing the intercellular connecting material with an enzyme, and smearing the free suspension onto the flat bottom of a culture dish such as a bottle or Petri dish to grow and proliferate the cells.

In this specification, the term “floating culture (suspension culture)” refers to the culture of cells in a culture medium in a floating state without fixing them to a substrate.

In the culture process, the cultured cells may be suspended in the medium by adding SR (serum replacement) or cultured using a low-adhesion culture dish or bioreactor, but they are not limited thereto. Various suspension culture methods known in the art may be used.

The cultures may be growth and proliferation.

In this specification, the terms “growth and proliferation” mean an increase in the number of cells. The culture may be undifferentiated proliferation. The undifferentiated proliferation refers to the proliferation of stem cells without differentiation into specific cells, but with the same properties as the original cells, that is, cells with potency and self-renewal capacity. In this specification, the term “differentiation” refers to the phenomenon in which the structure and function of cells become specialized during the division and proliferation of cells, meaning that the cells and tissues of living organisms change their forms or functions in order to perform their assigned tasks. Furthermore, differentiation may be confirmed by examining cell morphology using optical or confocal microscopy by measuring changes in cell surface markers (e.g., staining cells with tissue-specific or cell-marker-specific antibodies) and cell morphology (e.g., nucleus/cytoplasm ratio) using techniques such as flow cytometry or immunocytochemistry, or by measuring changes in gene expression using well-known techniques in the art, such as polymerase chain reaction (PCR) and gene-expression profiling.

In one embodiment, the medium composition may additionally contain amino acid mixtures or trace elements.

The types of amino acids included in the amino acid mixture are not limited and may include one or more of the following: alanine, cysteine, aspartic acid, glutamic acid, phenylalanine, glycine, histidine, isoleucine, lysine, leucine, methionine, asparagine, pyroglutamic acid, proline, glutamine, arginine, serine, threonine, selenocysteine, valine, tryptophan, and tyrosine.

The amino acid mixture may include one or more species selected from a group consisting of serine, aspartic acid, glutamic acid, alanine, glutamine, proline, tyrosine, valine and leucine.

In one embodiment, the medium composition may include an amino acid mixture at a concentration of 100 to 5000 mg/L, 100 to 4500 mg/L, 100 to 4000 mg/L, 100 to 3500 mg/L, 500 to 5000 mg/L, 1000 to 5000 mg/L, 1500 to 5000 mg/L, 2000 to 5000 mg/L, 2500 to 5000 mg/L, 500 to 4500 mg/L, 1000 to 4000 mg/L, 1500 to 3500 mg/L, or 2000 to 3000 mg/L.

The single-letter (three-letter) abbreviations for amino acids used in this specification refer to the following amino acids according to the standard abbreviation convention in the field of biochemistry: A (Ala): Alanine; C (Cys): Cysteine; D (Asp): Aspartic acid; E (Glu): Glutamic acid; F (Phe): Phenylalanine; G (Gly): Glycine; H (His): Histidine; I (Ile): Isoleucine; K (Lys): Lysine; L (Leu): Leucine; M (Met): Methionine; N (Asn): Asparagine; O (Ply): Pyroglutamic acid; P (Pro): Proline; Q (Gln): Glutamine; R (Arg): Arginine; S (Ser): Serine; T (Thr): Threonine; U (Sec): Selenocysteine; V (Val): Valine; W (Trp): Tryptophan; Y (Tyr): Tyrosine.

The trace elements refer to substances that are necessary for the growth of living organisms but only in very small amounts, and they are essential chemical elements that play a crucial role in the growth, development, and physiology of living organisms.

In one embodiment, the trace elements may be one or more selected from a group consisting of calcium (Ca), potassium (K), sodium (Na), iron (Fe), chlorine (Cl), boron (B), manganese (Mn), zinc (Zn), copper (Cu) and molybdenum (Mo).

In one embodiment, the medium composition may include trace elements at a concentration of 10 to 500 mg/L, 10 to 450 mg/L, 10 to 400 mg/L, 10 to 350 mg/L, 50 to 500 mg/L, 100 to 500 mg/L, 150 to 500 mg/L, 200 to 500 mg/L, 250 to 500 mg/L, 50 to 450 mg/L, 100 to 400 mg/L, 150 to 350 mg/L, or 200 to 300 mg/L.

In one embodiment, the medium composition include the amino acid mixture or trace elements at a concentration of 100 to 5000 mg/L, 100 to 4500 mg/L, 100 to 4000 mg/L, 100 to 3500 mg/L, 500 to 5000 mg/L, 1000 to 5000 mg/L, 1500 to 5000 mg/L, 2000 to 5000 mg/L, 2500 to 5000 mg/L, 500 to 4500 mg/L, 1000 to 4000 mg/L, 1500 to 3500 mg/L, or 2000 to 3000 mg/L.

In one embodiment, the basic medium may not include one or more vitamins selected from a group consisting of biotin, D-calcium pantothenate, a-ketoglutaric acid, choline chloride, folic acid, i-inositol, nicotinic acid, pyridoxine-HCl, riboflavin, thiamine-HCl, and penta-amino benzoic acid.

In one embodiment, the basic medium may not include vitamins.

In one embodiment, the basic medium may not include one or more organic acids selected from a group consisting of fumaric acid, malic acid, and succinic acid.

In one embodiment, the basic medium may not include organic acids.

In the case of vitamins and organic acids, if the cells are treated with an excessive amount, it can result in inhibiting cell proliferation.

Another aspect is to provide a method of preparing a cell line, comprising: separating cells from non-human animals; and suspension-culturing the separated cells.

In one embodiment, the method of preparing a cell line may not include a step of processing biological or chemical exogenous immortalization factors. Alternatively, the method of preparing a cell line may not include a step of inducing the immortalization of the cell by processing biological or chemical exogenous factors.

The biological exogenous immortalization factor or biological exogenous factor may be a vector incorporating muscle-specific and/or fat-specific growth genes.

For instance, the genes may be MyoD, SMARCD3, Pax3, Pax7, myosin-1, integrin alpha-7, cadherin-15, myogenin, growth hormones, insulin-like growth factor, myostatin, growth differentiation factor, and crustacean hyperglycemic hormone or myosin heavy chain gene.

The vector may be either an adenoviral vector or a lentiviral vector.

In addition, the biological exogenous immortalization factors or biological exogenous factors may be reprogramming microRNAs and mRNAS.

The reprogramming microRNAs may be mir302a-d, mir367, Oct4, Sox2, Klf4, c-Myc, or Lin28.

The chemical exogenous immortalization factor or chemical exogenous factor may be telomerase activators.

The chemical exogenous immortalization factor or chemical exogenous factor may be cycloastragenol, Genistein from Glycine Max (soybean), or resveratrol.

In one embodiment, the method of preparing a cell line may not include a step to process albumin or exogenous albumin.

In one embodiment, the cell may be one or more species selected from a group consisting of muscle stem cells, muscle cells, and progenitors thereof.

The muscle stem cells may be, but are not limited to, satellite cells or myoblasts, e.g., myoblasts.

The muscle stem cells are as described above.

The muscle stem cells may be cells that exhibit expression of Pax7 or MyoD in the process of being induced into myoblasts.

The myoblasts are as described above.

The muscle cells are as described above.

The muscle cells may be, but are not limited to, myocytes or myotubes, e. g., myocytes.

The progenitor cells refer to cells capable of producing cells differentiated into multiple lineages, e.g., myoblasts, fibroblasts, adipocytes, stromal cells, epithelial cells, smooth muscle cells, and endothelial cells. Progenitor cells differ from stem cells in that they typically do not have the ability to self-replicate extensively.

The animal cells may include, but are not limited to, fat cells (fat cells or adipocytes) and/or progenitor cells thereof, stromal cells (connective tissue) and/or progenitor cells thereof, or endothelial cells (blood vessels) and/or progenitor cells thereof.

In one embodiment, the non-human animal may be cattle, sheep, pigs, poultry, crustaceans, or fish.

In one embodiment, the crustaceans may be shrimps, crabs or lobsters.

In one embodiment, the suspension culture may be performed at 100 to 1000 RPM, 100 to 900 RPM, 100 to 800 RPM, 100 to 700 RPM, 100 to 600 RPM, 100 to 500 RPM, 100 to 400 RPM, 100 to 300 RPM, 200 to 1000 RPM, 300 to 1000 RPM, 200 to 900 RPM, 200 to 800 RPM, 200 to 700 RPM, 200 to 600 RPM, 200 to 500 RPM, 200 to 400 RPM, 250 RPM, or 300 RPM.

In one embodiment, the suspension culture may be performed for a period of 1 to 42 days, 1 to 35 days, 1 to 28 days, 7 to 42 days, 14 to 42 days, 21 to 42 days, 7 to 35 days, 14 to 28 days, 21 to 28 days, or 28 days.

In one embodiment, the suspension culture may be performed at a temperature of 10 to 50 °C, 10 to 40 °C, 10 to 30 °C, 20 to 50 °C, 25 to 50 °C, 20 to 40 °C, 25 to 30 °C or 28 °C

In one embodiment, the suspension culture may be performed in serum-free medium.

In one embodiment, the suspension culture may be performed in a medium containing serum.

In this specification, the term “suspension culture” refers to the culture of cells in a culture medium in a floating state without fixing them to a substrate.

In this specification, the terms “suspension culture” and “floating culture” are synonymous.

In this specification, the terms “static culture” and “adhesive culture” are synonymous.

In the culture process, the cultured cells may be suspended in the medium by adding SR (serum replacement) or cultured using a low-adhesion culture dish or bioreactor, but they are not limited thereto. Various suspension culture methods known in the art may be used.

The cultures may be growth and proliferation.

The growth and proliferation are as described above.

In one embodiment, the culture media may be, but are not limited to, one or more species selected from a group consisting of DMEM (Dulbecco's Modified Eagle's Medium), MEM (Minimal Essential Medium), BME (Basic medium Eagle), RPMI 1640, DMEM/F-10 (Dulbecco's Modified Eagle's Medium: Nutrient Mixture F-10), DMEM/F-12 (Dulbecco's Modified Eagle's Medium: Nutrient Mixture F-12), a-MEM (a-Minimal Essential Medium), G-MEM (Glasgow′ s Minimal Essential Medium), IMDM (Isocove's Modified Dulbecco's Medium), Knockout DMEM, E8 (Essential 8 Medium), SF-DMEM (serum free-Dulbecco Modified Eagle Medium), L15 Medium, and Grace's Insect Medium.

In addition to the aforementioned culture media, the media used in the industry are sufficient, and may contain amino acids, vitamins, mineral salts, and other ingredients. For example, non-essential amino acids and L-glutamine, vitamins such as vitamin B12, inorganic salts such as trace components (CuSO45H20, Fe(NO3)39H20, ZnSO4), phosphoenol pyruvate, monoethanolamine, sodium selenite and sodium bicarbonate, and other ingredients such as d-glucose, linoleic acid. Linoleic acid, lipoic acid, sodium pyruvate, hypoxantine Na, putrescine HCl, and polyamine solutions may be included.

In one embodiment, the culture media may be a serum-free medium.

A serum-free medium refers to a cell culture medium that does not use serum, which has ethical, environmental, and high-cost issues, and eliminates the use of serum.

A different aspect is to provide a cell line prepared by a method of preparing a cell line, comprising separating a cell from a non-human animal; and suspension-culturing the separated cell.

In one embodiment, the cell line may be, but not limited to, capable of maintaining cell proliferation capacity for 1 to 12 months at −200 to −75 °C.

A different aspect is to provide a method of culturing a cell line, comprising performing suspension culture for proliferation of a cell line prepared by the method of preparing a cell line.

The RPM and temperature at which the suspension culture for the proliferation of the cell line can be performed are as described above.

In one embodiment, the suspension culture for proliferation of a cell line may be performed in a medium containing serum.

The serum may be, but is not limited to, animal blood, is preferably serum derived from mammalian blood (e.g., pig, horse, cow, goat, sheep, and dog blood), and more preferably FBS.

In one embodiment, the serum may be, but is not limited to, one or more species selected from a group consisting of fetal bovine serum (FBS), human platelet lysate (hPL), human serum albumin (HSA), human serum (HS), platelet-rich plasma (PRP), platelet poor plasma (PPP), calf serum, horse serum, and pig serum (porcine serum), and sheep serum.

In one embodiment, the serum may be included in an amount of 1 to 20 wt %, 3 to 18 wt %, 5 to 16 wt %, 7 to 14 wt %, 9 to 12 wt %, or 10 wt % based on the total weight of the medium.

Another aspect is to provide cultured meat comprising a cell line prepared by a method of preparing a cell line, comprising separating a cell from a non-human animal and suspension-culturing the separated cell.

In this specification, the term “cultured meat” is also referred to as a clean meat or cell-based meat or cultivated meat, and means that it is suitable for human or non-human animal consumption or is edible.

In one embodiment, a food composition comprising the cultured meat is provided.

The food may be one or more types selected from a group consisting of snacks, dumplings, fried foods, stir-fries, steamed foods, sauces, seasonings, powder mixes, breads, processed stewed foods, beverages, dried seaweed, processed noodles and processed foods.

The form of the cultured meat added to food may be in the form ground into various particle sizes, depending on the purpose of use in the food. It may be uniformly or unevenly ground in the range of 1 μm to 10 cm and added to food. Alternatively, it may be added in the form of a dried powder, depending on the purpose of use in the food.

The food composition may further include fats and/or colorants. The fat may be injected into the fat cells together in the process of preparing a cultured meat and included in the process of proliferating muscle cells by co-culture.

Alternatively, it may be included by adding liquid fat. In this case, saturated fatty acids in meat can be replaced with beneficial fats, which can be good for health.

In one embodiment, fats may include vegetable oils, such as soybean oil, corn oil, canola oil, rice bran oil, sesame oil, extracted sesame oil, perilla oil, extracted perilla oil, safflower oil, sunflower oil, cottonseed oil, peanut oil, olive oil, palm oil, coconut oil, chili seed oil, animal oils and fats, such as edible beef tallow, edible pig fat, raw beef fat, raw pork fat, fish oil, and processed edible oils and fats, such as mixed edible oil, flavored oil, processed oil, shortening, margarine, imitation cheese, and vegetable cream.

Colorants refer to compounds that give color to food, and may include, but are not limited to, artificial colorants, natural colorants, and natural extracts (e. g., beet roots extract, pomegranate fruit extract, cherry extract, carrot extract, red cabbage extract, and red seaweed extract), modified natural extracts, natural juices (e.g., beet root juice, pomegranate juice, cherry juice, carrot juice, red cabbage juice, and red seaweed juice), modified natural juices, FD&C (Food Drug & Cosmetics) red 3 (erythrosin), FD&C green 3 (fast green FCF), FD&C red 40 (allura red AC), FD&C yellow 5 (tartazine), FD&C yellow 6 (sunset yellow, FCF), FD&C blue 1 (brilliant blue, FCF), FD&C blue 2 (indigotine), titanium oxide, annatto, anthocyanins, beta-APE 8 carotenal, beta-carotene, black currant, burnt sugar, canthaxanthin, caramel, carmine/carminic acid, cochineal extract, curcumin, lutein, carotenoids, monascin, paprika, riboflavin, saffron, turmeric, and any combination thereof, to reproduce the red flesh color of beef or pork. In addition, color formers, such as nitrite, and ascorbic acid, erysobric acid, or salts thereof, which promote the color development of the nitrite, may be further added as color development aids.

In addition, antioxidants, emulsifiers, and salts may be added to stabilize proteins to prevent rancidity, color change, or separation of fats. The antioxidants, emulsifiers, salts, etc., can be used without restriction as long as they are widely used in the industry.

### Advantageous Effects of the Invention

By a medium composition for animal cell culture, comprising yeast extract or peptone, according to an aspect, the growth and proliferation of animal cells is possible even in a medium that does not contain serum, and the growth and proliferation of animal cells can be promoted. By a medium composition for animal cell culture, comprising a basic medium and yeast extract, cells can effectively be proliferated even in the basic medium that does not contain vitamins and organic acids, thereby reducing the cost of cell culture media by minimizing the ingredients of the culture medium. By a method of preparing a cell line, comprising separating a cell from a non-human animal; and suspension-culturing the separated cell, according to an aspect, there is an effect to obtain a cell line effectively from a non-human animal.

### BRIEF DESCRIPTION OF THE DRAWINGS

FIG. 1 shows the difference in cell proliferation depending on the culture medium used for animal cell culture.

FIGS. 2 to 4 show the difference in the proliferation of animal cells by culturing animal cells in a medium containing peptone and comparing them with or without the addition of FBS in a medium.

FIGS. 5 to 7 show the difference in the proliferation of animal cells by culturing animal cells in a medium containing yeast extract and comparing them with or without the addition of bovine serum in a medium.

FIG. 8 shows the results of measuring the consumption of amino acids over time when animal cells are cultured in a medium containing yeast extract.

FIGS. 9 to 14 show the results of observing the proliferation of animal cells by additionally adding an amino acid mixture to yeast extract or peptone containing a medium.

FIGS. 15 to 20 show the results of observing the proliferation of animal cells by additionally adding trace elements to a medium containing yeast extract or peptone.

FIG. 21 shows the difference in cell proliferation with or without the inclusion of vitamins in the basic medium.

FIG. 22 shows the difference in cell proliferation with or without the inclusion of organic acids in the basic medium.

FIG. 23 shows the difference in cell proliferation with or without the inclusion of vitamins and organic acids in the basic medium.

FIGS. 24 and 25 are schematics showing the process of establishing a cell line from crustaceans.

FIG. 26 shows the results of observing cells separated from crustaceans with a microscope.

FIGS. 27 to 29 show the results of culturing cells separated from crustaceans under different culture conditions and identifying the cell proliferation.

FIG. 30 shows the results of identifying whether the cells separated from crustaceans have been established as cell lines and have sustained proliferation capacity even after long-term culture.

FIG. 31 shows the results of identifying whether the cells separated from crustaceans have been established as cell lines and have sustained proliferation capacity even after long-term storage.

### IMPLEMENTATIONS OF THE INVENTION

The following examples provide a more detailed description of the invention. It should be noted, however, that these examples are intended to be illustrative and not limit the scope of the present invention.

The terms or words used in the specification and claims of the present invention shall not be limited or construed in their ordinary or dictionary senses, but shall be construed in a sense and concept consistent with the technical idea of the present invention based on the principle that the inventor may properly define the concept of the term in order to best describe his or her invention.

In the context of this specification as a whole, when a part is said to “comprise,” “include,” or “contain” a component, it means that it may include more other components, rather than excluding other components, unless otherwise stated to the contrary.

Throughout this specification, “A and/or B” means A or B, or A and B.

### Example 1. Checking Cell Proliferation by Medium

In order to determine whether there was a difference in cell proliferation when different culture media were used, crustacean-derived cells separated from shrimp were cultured using a commonly used basic medium (DMEM-F12, L15, or Grace's insect media) with an addition of 0, 5, or 10% (v/v) FBS, and the extent of cell proliferation was evaluated.

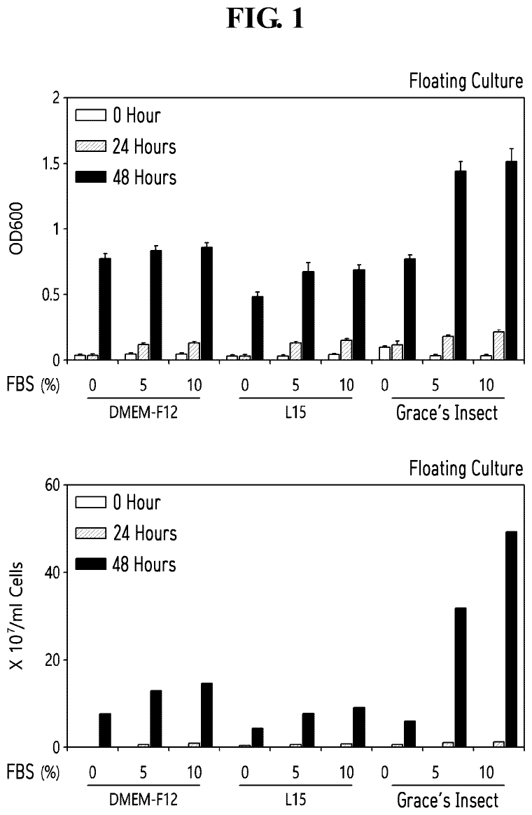

Specifically, crustacean cells were initially inoculated at a density of 1.5×10⁵ cells/ml under conditions at 300 rpm at 28 to 32 °C, and suspension-cultured for 48 hours, and the proliferation of cells in three basic media was compared. To assess the overall tendency of cell proliferation, the absorbance at OD600 was measured, and the exact number of cultured cells was counted using a hemocytometer. The results are presented in FIG. 1.

As shown in FIG. 1, the results of the absorbance measurement and cell number count using a hemocytometer confirmed the highest cell proliferation in Grace's insect media. In addition, the cell proliferation was higher in the media with the addition of FBS than in the culture medium without the addition of FBS. From the above results, it was found that the proliferation rate of crustacean cells significantly varies depending on the type of basic medium, even when the same FBS is included, and it was also found that while Grace's insect media is suitable for the proliferation of crustacean cells, cell proliferation can still occur even when FBS is added to other basic culture media.

### Example 2. Cell Proliferation in Peptone-Containing Media

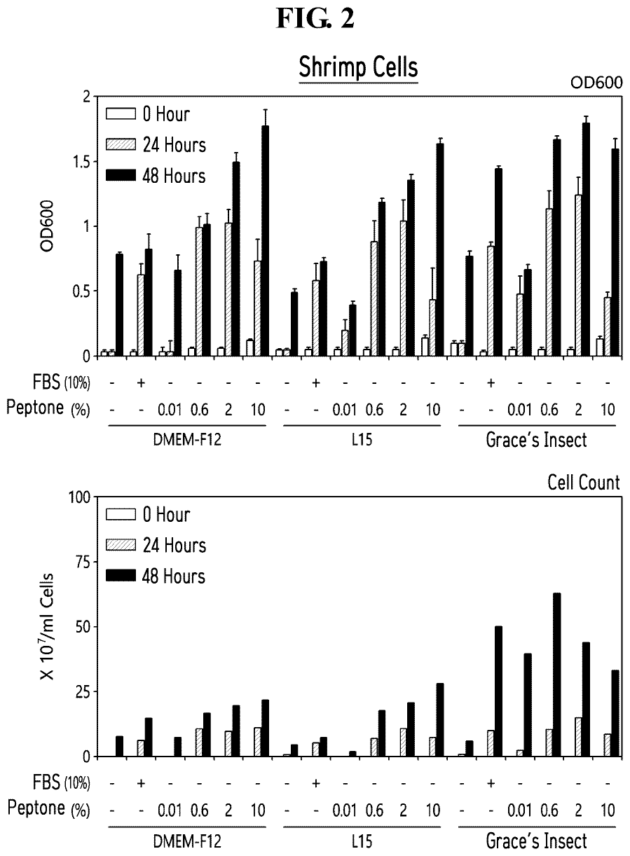

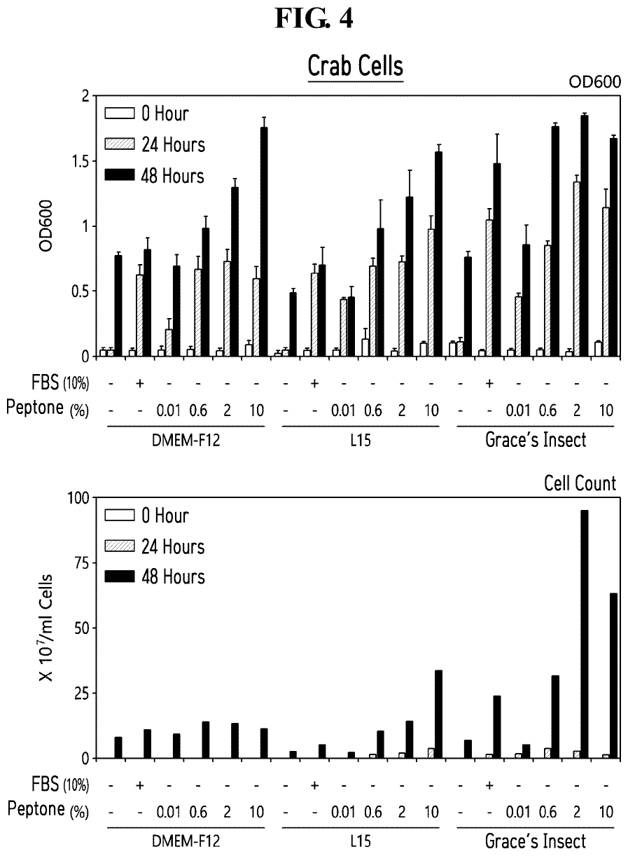

In order to determine whether peptone can be used as a substitute for FBS, which has a cell proliferation effect, in the culture of animal cells, the cell proliferation effect of peptone, which is not commonly used in animal cell culture, was evaluated. Peptone is a generic term for products produced during enzymatical decomposition or hydrolysis of various proteins, and it includes a mixture of components such as polypeptides, amino acids, and minerals. Specifically, the culture medium was prepared by adding peptone to the three basic media types (DMEM-F12, L15 or Grace's insect media) to the concentration of 0.01, 0.6, 1 or 10% (w/v) , and the proliferation of crustacean-derived cells separated from shrimp, crab, or lobster was evaluated by comparing it to the culture medium with or without FBS, in the same way as Example 1. The results are presented in FIGS. 2 and 4.

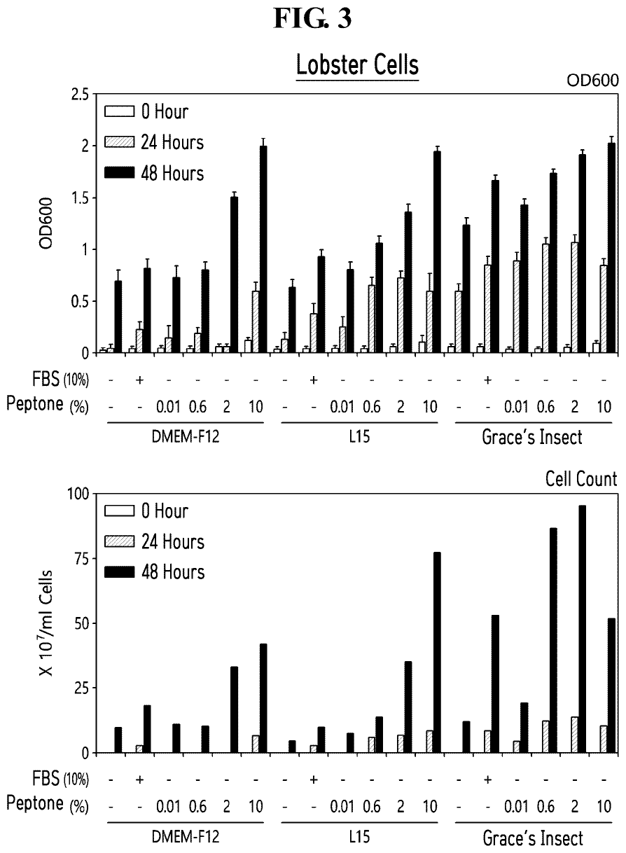

As shown in FIGS. 2 to 4, very insignificant cell proliferation was found when FBS and peptone were not added to DMEM-F12, L15 or Grace's insect media. However, when peptone was added to these three basic media to culture cells, the cell proliferation rate was higher than when 10% (v/v) FBS was included, and the highest cell proliferation was observed when peptones were added to Grace's insect media among the three. Furthermore, when peptone was added to all basic media, the cell proliferation rate was increased compared to the culture medium with 10% (v/v) FBS, confirming that peptone was more effective in cell proliferation than FBS.

### Example 3. Cell Proliferation in Yeast Extract-Containing Media

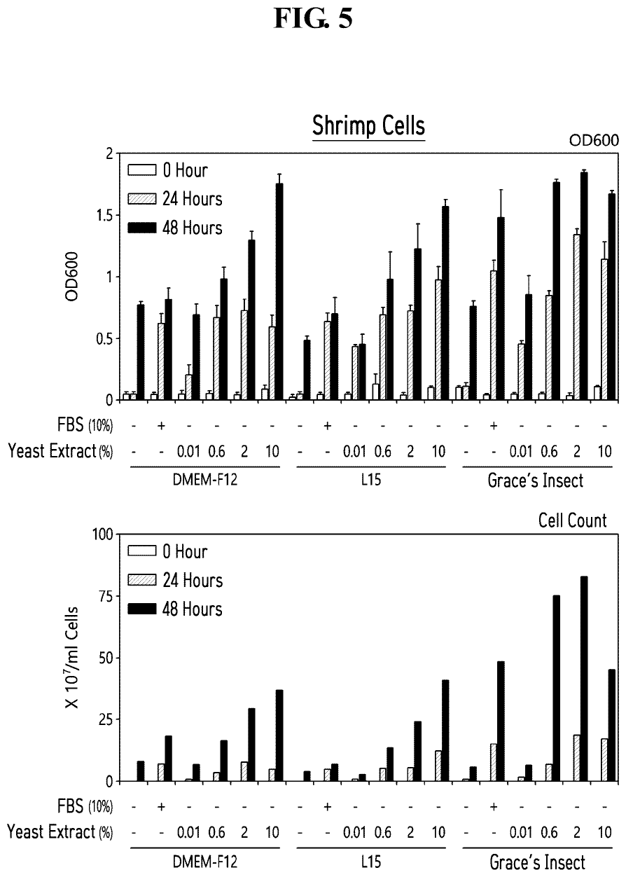

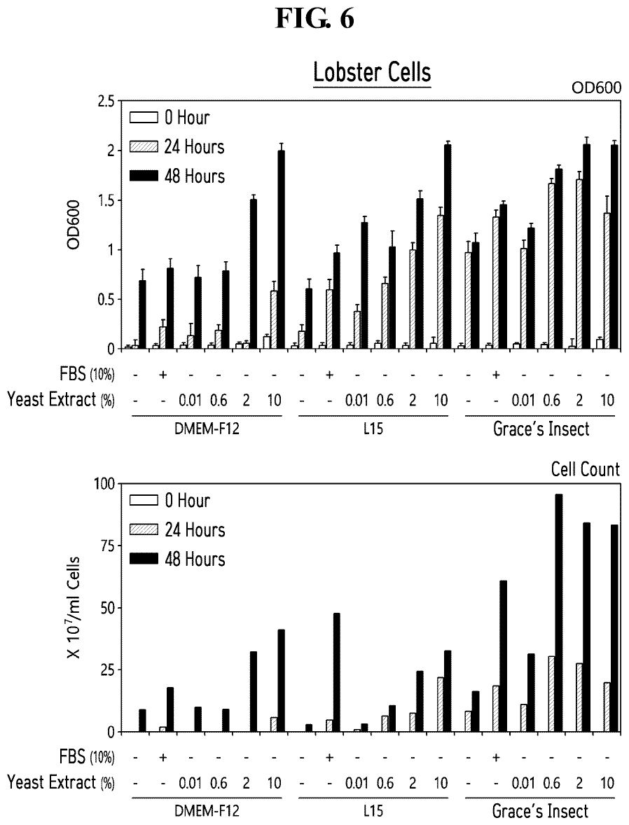

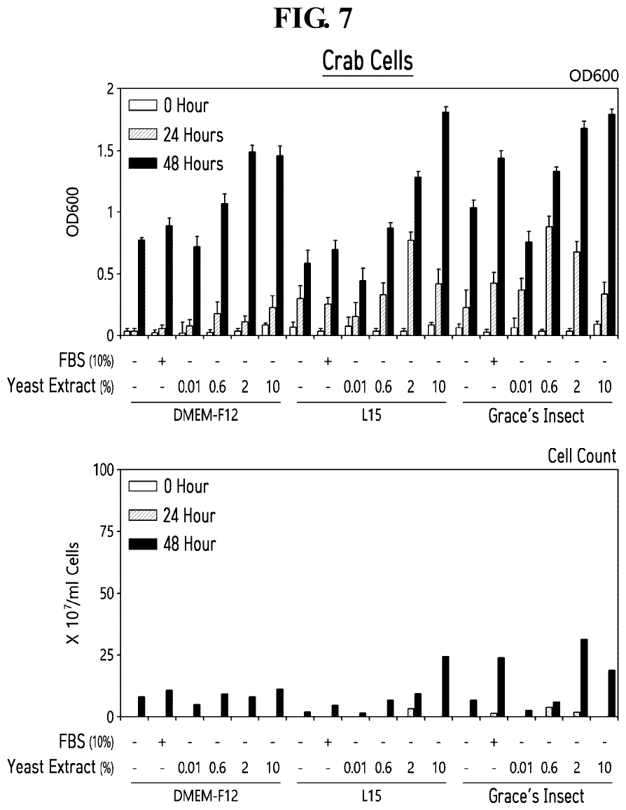

In order to determine whether yeast extract can be used as a substitute for FBS, which has a cell proliferation effect, in the culture of animal cells, whether the addition of yeast extract showed any changes in cell proliferation was evaluated. Except for the use of yeast extract instead of peptone, it was confirmed by the same method as Example 2. The results are shown in FIGS. 5 to 7.

As shown in FIGS. 5 to 7, the highest cell proliferation rate was observed when Grace's insect media was used as the basic medium and yeast extract was added. From the above results, it was confirmed that the addition of yeast extract to the medium was more effective in cell proliferation than the medium with the addition of FBS.

### Example 4. Measurement of Amino Acid Consumption in Cell Culture and Cell Proliferation in Amino Acid Mixture-Added Media

### 4.1. Measurement of Amino Acid Consumption

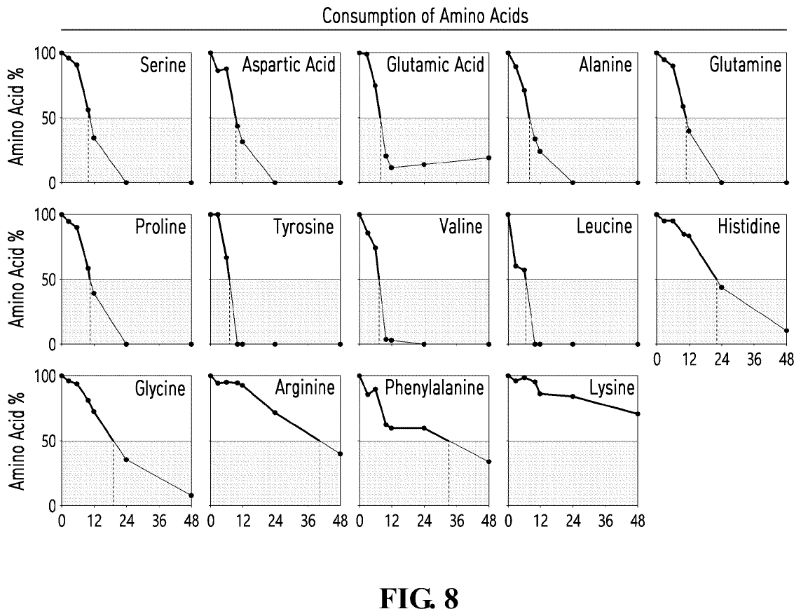

After confirming that the culture medium with yeast extract added to Grace's insect media was effective in cell proliferation, in order to find out how to further increase the cell proliferation rate, the consumption of amino acids during the cell culture process was measured using ultra performance liquid chromatography (UPCL). Specifically, shrimp cells were cultured in culture medium with 0.6% (w/v) yeast extract added to Grace's insect media, and amino acid consumption was measured by collecting the culture medium for 3, 6, 10, 12, 24, and 48 hours after cell culture. The results are shown in FIG. 8.

As shown in FIG. 8, a total of nine amino acids, including serine, aspartic acid, glutamic acid, alanine, glutamine, proline, tyrosine, valine, and leucine, were found to be consumed by more than 50% within 12 hours.

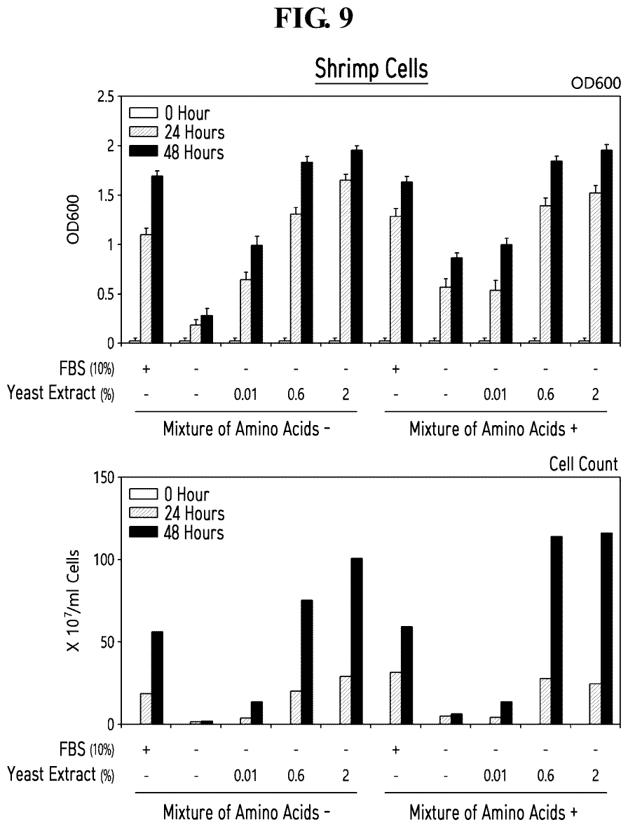

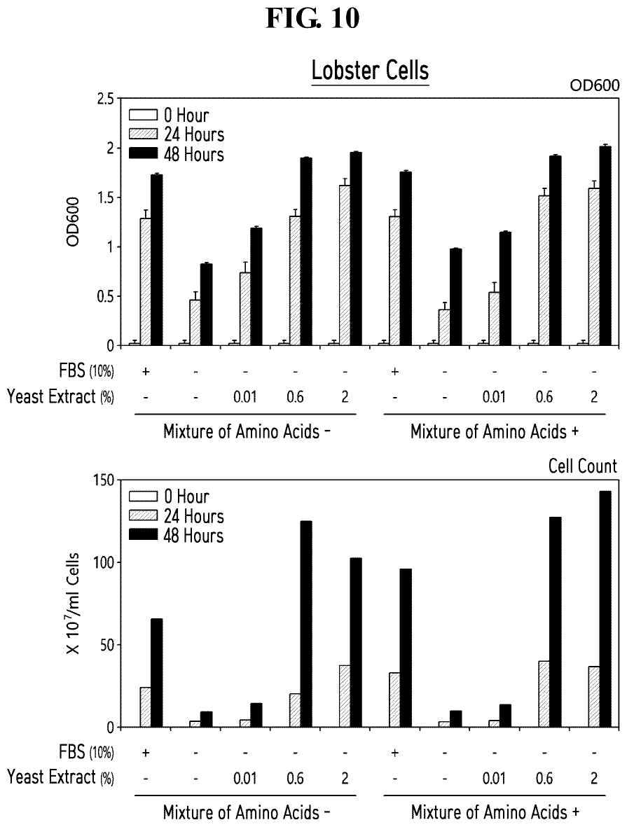

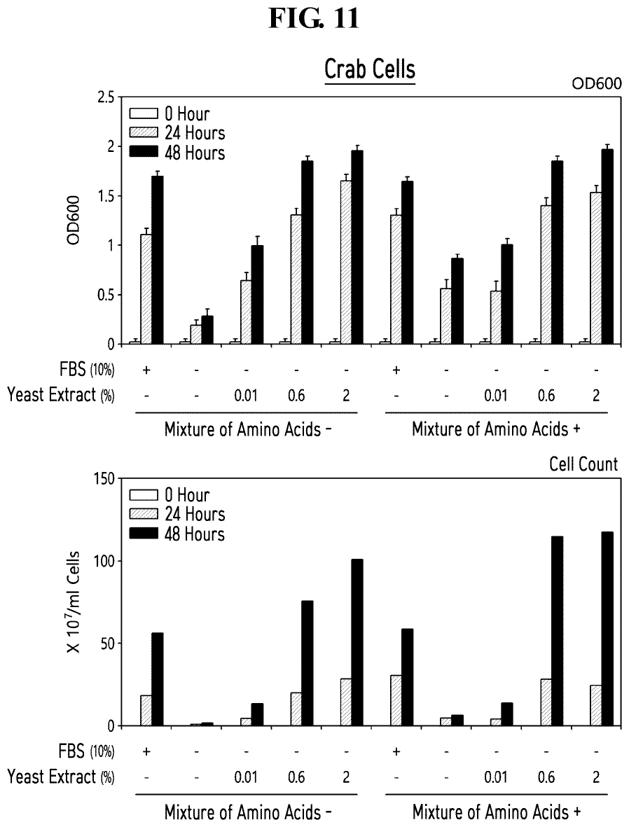

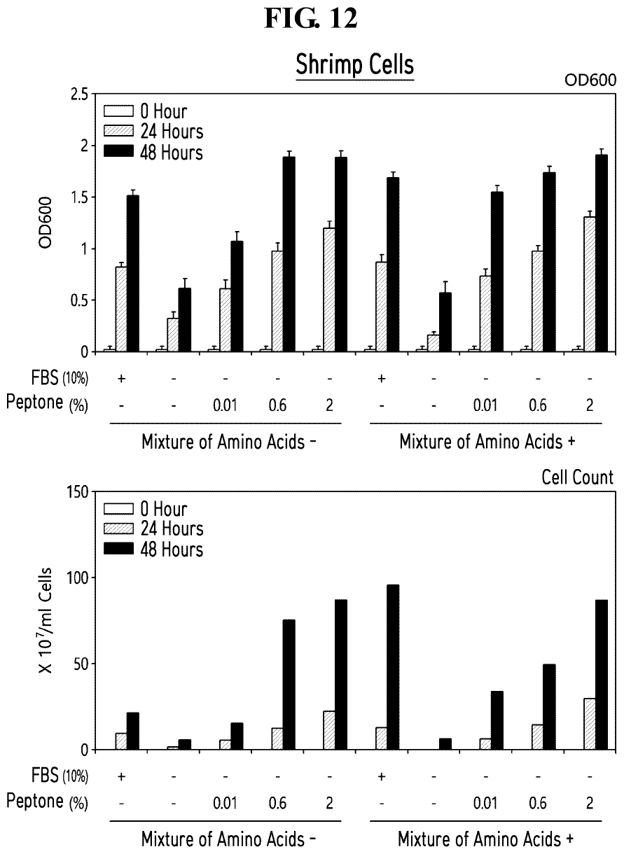

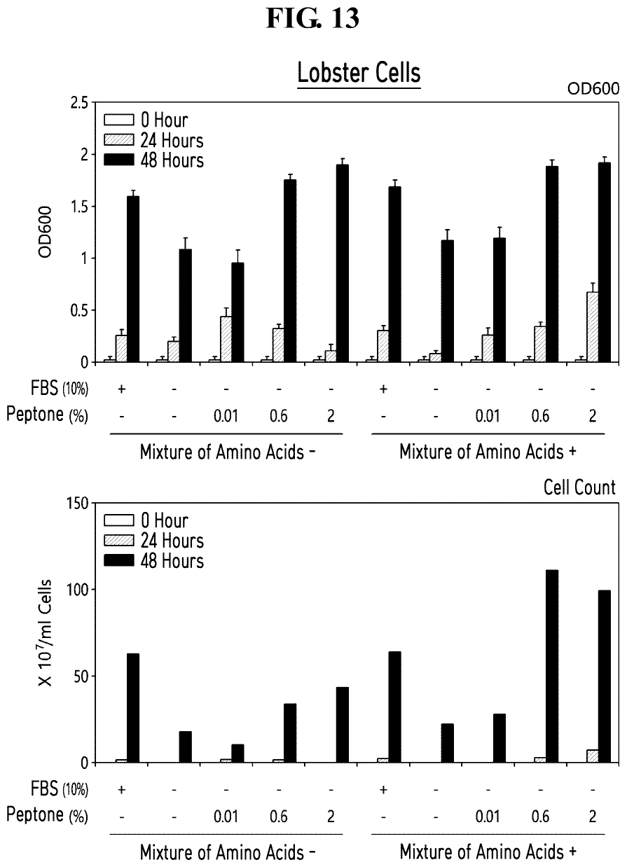

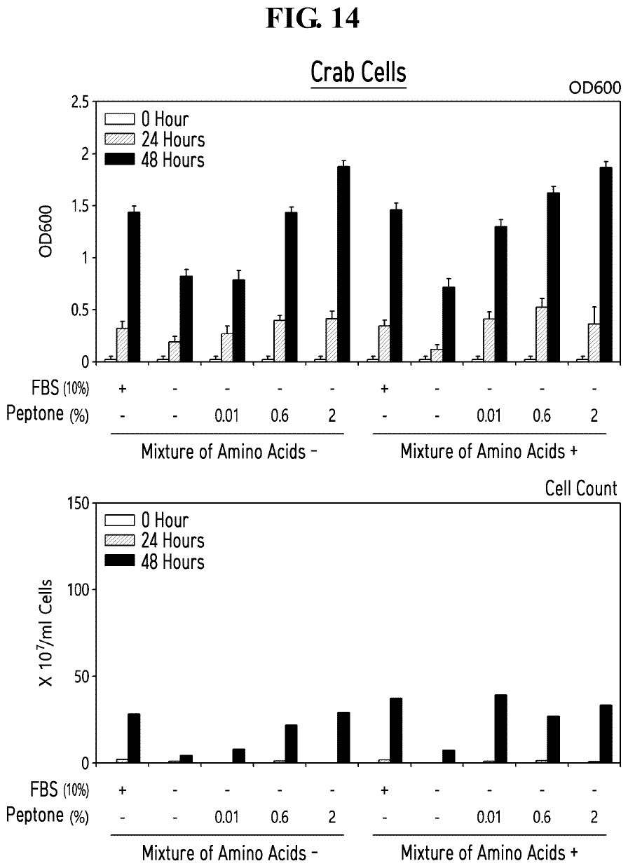

4.2. Cell Proliferation in Amino Acid Blended Media Assuming that more than 50% of the amino acids consumed within 12 hours are used at the beginning of 41 cell proliferation and have a close effect on the cell proliferation rate, the cell proliferation by an addition of the amino acid mixture were observed by adding a mixture of the nine amino acids that were consumed first to Grace's insect media with yeast extract or peptone. Specifically, the amino acid mixture was added to the concentration of alanine 225 mg/L, aspartic acid 350 mg/L, glutamic acid 600 mg/L, glutamine 600mg/L, leucine 75 mg/L, proline 350 mg/L, serine 1100 mg/L, tyrosine* 275 mg/L, and valine 100 mg/L, and cell proliferation was observed. The results are shown in FIGS. 9 to 14.

As shown in FIGS. 9 to 14, significantly faster cell proliferation was observed in culture media with the nine amino acids than without the nine amino acids, 24 hours after cell culture began, and after 48 hours from the beginning of cell culture, it was observed that more cells proliferated in culture media with the amino acid mixture than in cell culture media without the nine amino acids. From the above results, it can be learned that the addition of amino acids with high initial consumption during cell culture increases the rate of cell proliferation and the amount of proliferation.

### Example 5. Cell Proliferation in Trace Elements-Added Media

Yeast extract consists primarily of amino acids, vitamins, and trace elements. Trace elements are substances that are essential for the growth of living organisms, but are only needed in very small amounts, and are essential chemical elements that play an important role in the growth, development, and physiology of living things. These include mercury, cadmium, iodine, and most other heavy metals.

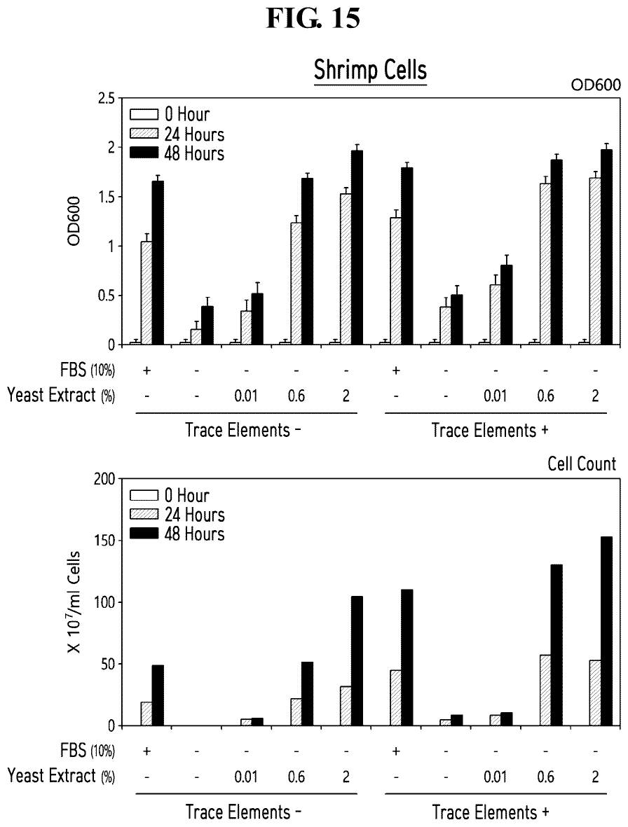

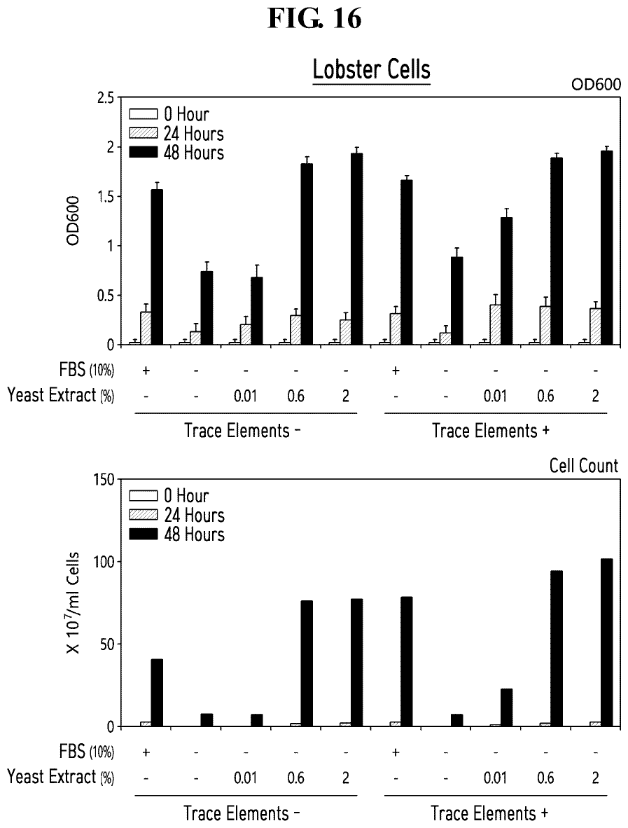

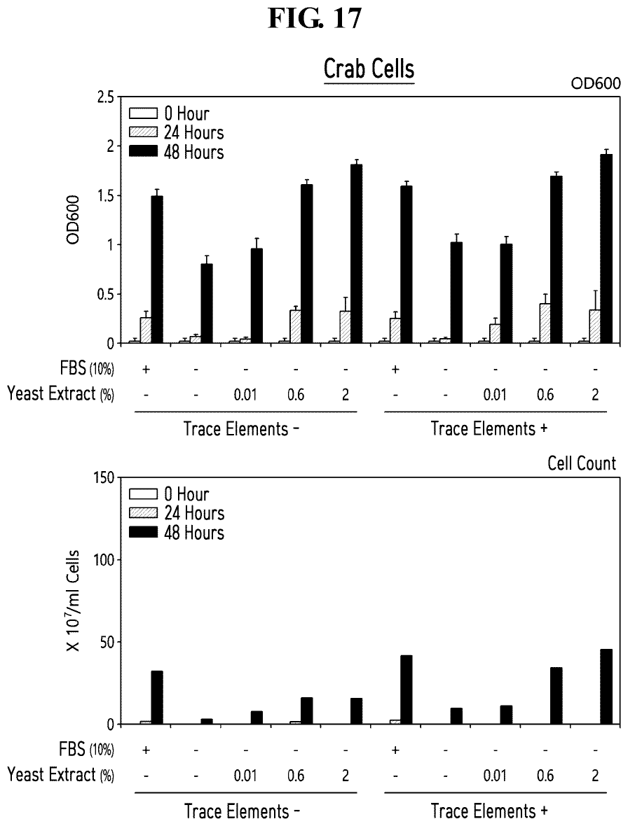

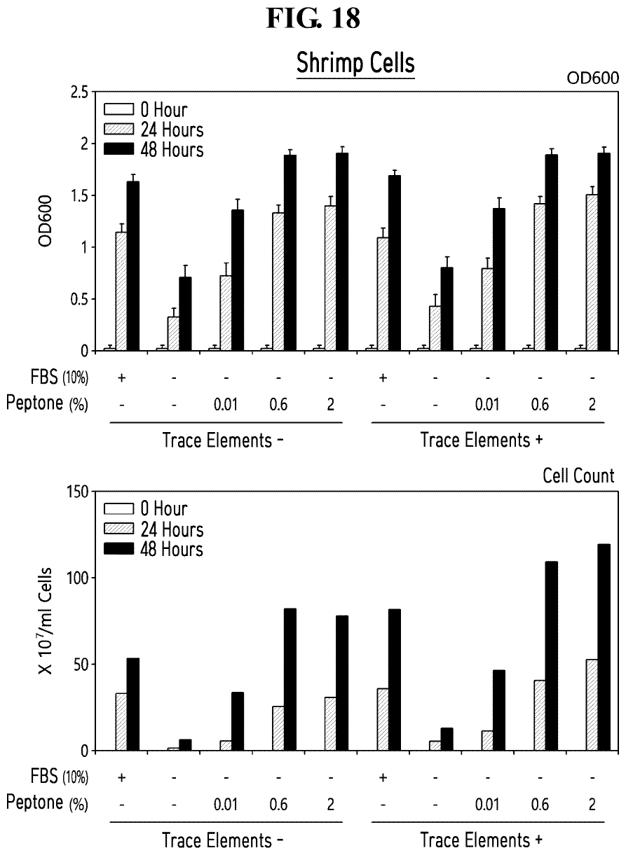

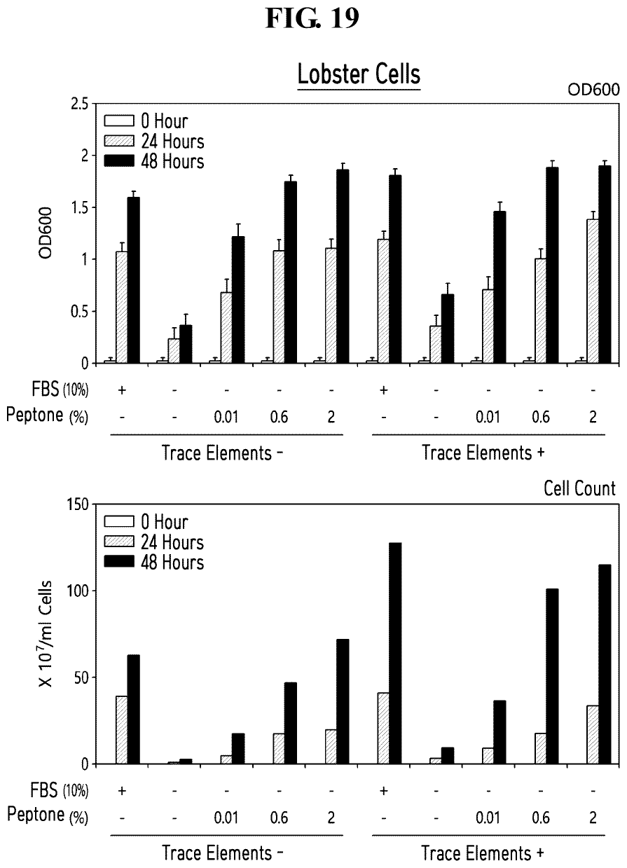

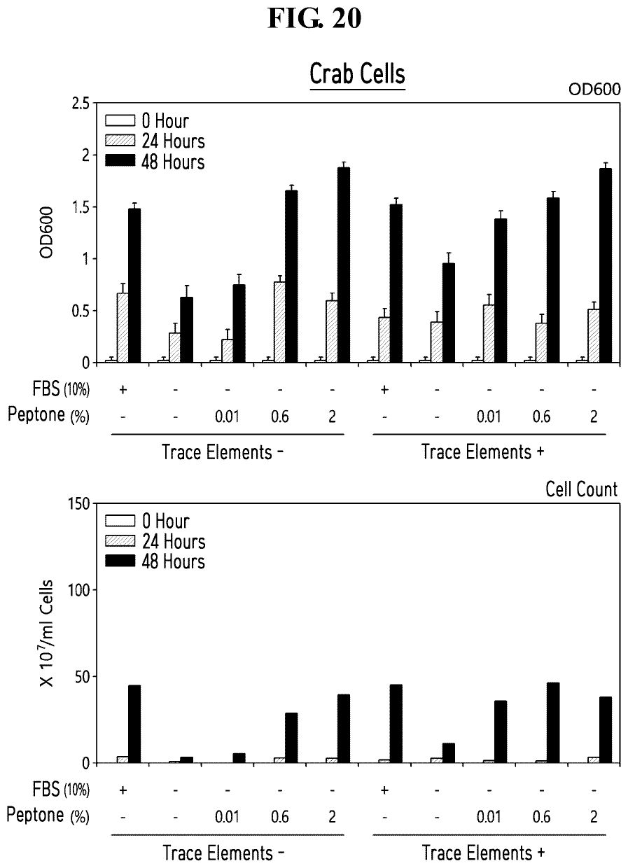

In order to identify substances that can promote cell proliferation in addition to the nine amino acids, the amount of trace elements contained in yeast extract was analyzed, trace elements were additionally added to the culture medium containing yeast extract or peptone (Grace's insect media+yeast extract+trace elements or Grace's insect media+peptone+trace elements) and cell proliferation experiments were performed to see how they affect cell proliferation. Specifically, trace elements were added at concentrations of calcium 5.28 mg/L, magnesium 16.2 mg/L, potassium 258 mg/L, sodium 50.4 mg/L, zinc 0.084 mg/L, copper 0.0414 mg/L, and manganese 0.021 mg/L, and cell proliferation was observed. The results are shown in FIGS. 15 to 20.

As shown in FIGS. 15 to 20, it was found that the rate of cell proliferation and the amount of final proliferation were increased when trace elements were added to the shrimp cell culture medium compared to the control group without the addition of trace elements, and identically as in shrimp cells, when trace elements were added to the crab and lobster cell culture media, the rate of cell proliferation and the amount of final proliferation were increased compared to the control group without the addition of trace elements. As a result of the above, it was confirmed that the addition of trace elements to the crustacean cell culture medium helps to increase the rate of cell proliferation and the amount of final proliferation.

### Example 6. Cell Proliferation With or Without the Inclusion of Vitamins in the Basic Medium

Yeast extract is an extract obtained by extracting the water-soluble components of yeast and removing moisture, including amino acids, peptides, carbohydrates, organic acids, vitamins, and minerals. Similar to yeast extracts, the basic media used in cell culture is composed of amino acids, sugars, organic acids, vitamins, organic acids, and minerals, so the composition is similar to that of yeast extract, but the content is different.

Yeast extract was added to the basic medium with or without vitamins and cells were cultured in order to determine if the presence of vitamins in a basic medium affects cell proliferation and if the vitamin components in the basic medium can be replaced with vitamin found in yeast extract alone.

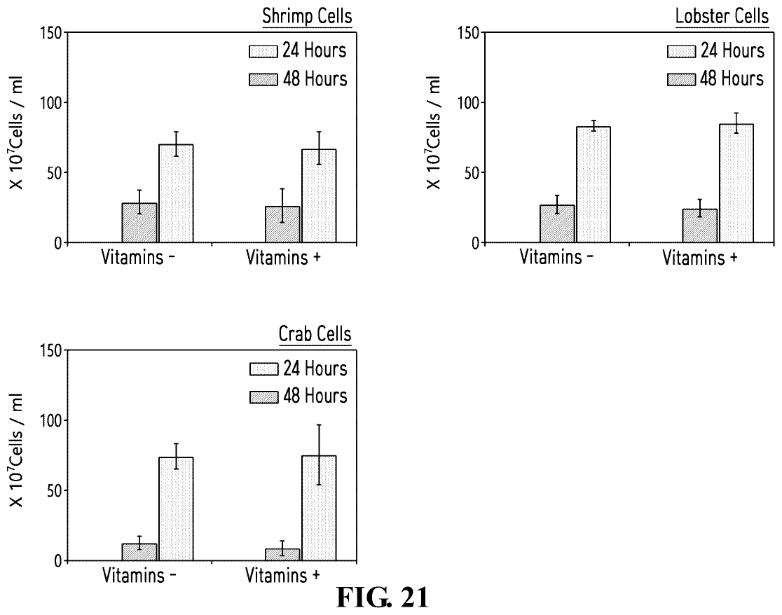

Specifically, a medium composition in which yeast extract was added at a concentration of 0.6% (w/v) to a basic medium (Grace's insect media, serum free) excluding vitamin components and a media composition in which yeast extract was added to the basic medium at a concentration of 0.6% (w/v) were prepared and the cell proliferations were compared. Under conditions at 300 rpm at 28 to 32 °C, crustacean cells were inoculated initially at 1.5×10⁵ cells/ml and suspended for 48 hours. Cells were collected every 24 hours to check whether cells proliferated through a hemocytometer. The results are shown in FIG. 21.

As shown in FIG. 21, neither crustacean cells showed a difference in cell proliferation with or without the addition of vitamins. Through the above results, it was confirmed that the vitamin components of yeast extract alone are effective in the proliferation of crustacean cells, so it was experimentally confirmed that the vitamin components of yeast extract can replace the vitamin component of the basic culture medium (Grace's insect media).

### Example 7. Cell Proliferation With or Without the Inclusion of Organic Acids in the Basic Medium

Yeast extract was added to the basic medium with or without organic acids and cells were cultured, in order to determine if the presence of organic acids in a basic medium affects cell proliferation and if the organic acid components in the basic medium can be replaced with the organic acid found in yeast extract.

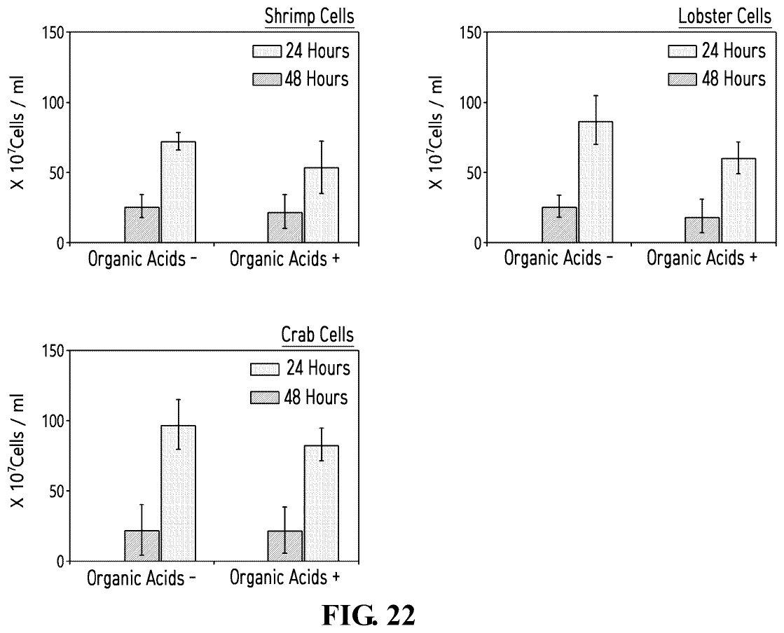

Specifically, it was observed how crustaceans cell proliferation changed in a media composition in which yeast extract was added to a basic medium (Grace's insect medium, serum free) at a concentration of 0.6% (w/v) and in a media composition in which fumaric acid, malic acid, and succinic acid were removed from the basic medium and yeast extract was added at a concentration of 0.6% (w/v). The proliferations of cells were compared in the same way as in Example 6, and the results are shown in FIG. 22.

As shown in FIG. 22, it was found that the media composition without the addition of fumaric acid, malic acid, and succinic acid was more effective in cell proliferation than the medium composition with the addition of fumaric acid, malic acid, and succinic acid, while fumaric acid, malic acid, and succinic acid were found to inhibit crustacean cell proliferation. From the above results, it was possible to derive the result that the organic acid components contained in the yeast extract alone are effective in cell proliferation, and that the organic acid added as a component of the basic medium inhibits cell proliferation.

### Example 8. Cell Proliferation with or without the Inclusion of Vitamins and Organic Acids in the Basic Medium

Yeast extract was added to the basic medium with or without vitamins and organic acids and cell were cultured in order to determine if the presence of vitamins and organic acids in the basic medium affects cell proliferation and if the vitamin and organic acid components in the basic medium can be replaced with the vitamin and organic acid found in yeast extract.

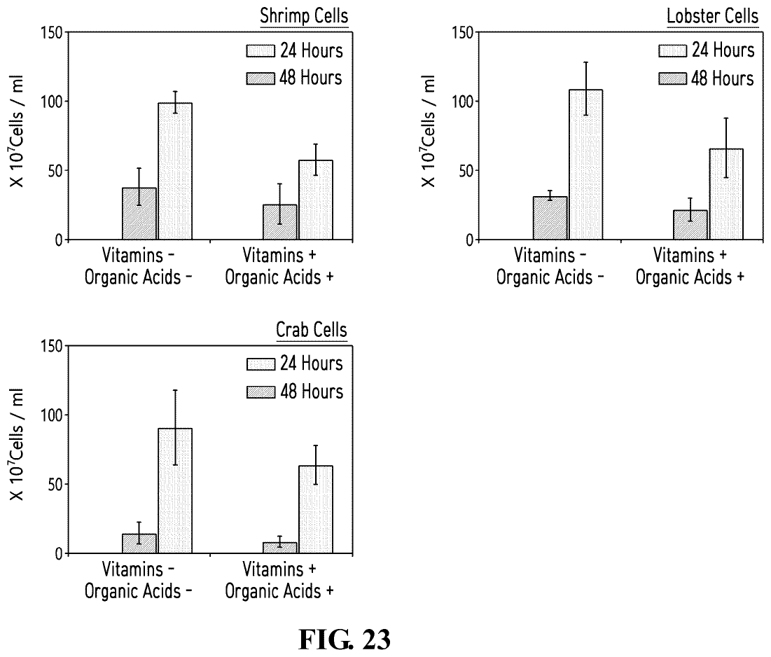

Specifically, a media composition in which yeast extract was added to a concentration of 0.6% (w/v) in a basic medium (Grace's insect media, serum-free) and a media composition in which vitamins and organic acids were removed and yeast extract was added to a concentration of 0.6% (w/v) in the basic medium were prepared, and crustacean cells were cultured to confirm cell proliferation. The proliferations of cells were compared in the same way as Example 6, and the results are shown in FIG. 23.

As shown in FIG. 23, cells cultured in a medium composition without the addition of vitamins and organic acids showed faster and higher proliferation than cells cultured in a medium composition with added vitamins and organic acids, and it was confirmed that cell proliferation was inhibited by organic acids as shown in the results of Example 4.

Combining the above results, it was found that the vitamins contained in the yeast extract can replace the vitamins contained in the basic medium, and that the exclusion of the vitamin and organic acid components contained in the basic medium is effective for the proliferation of crustacean cells, since the addition of organic acid components in the basic medium in addition to the organic acids in the yeast extract inhibits cell proliferation.

### Example 9. Separation of Cells From Shrimp and Establishment of Cell Lines

The live shrimp were stunned by placing them on ice for 15 minutes before sacrificing them. The body segments were cleaned with a brush under running tap water and sterilized by soaking them in electrolytic sodium hypochlorite water (NaOCl) for 5 minutes three times. After cleaning with sterile distilled water, the head and tail were removed. After breaking the segments of the body, the internal organs were removed by piercing the segments with pins, and then all the skin was removed. It was sterilized by soaking in electrolytic sodium hypochlorite water for 5 minutes, and then washed with sterilized distilled water. Body tissue sections were washed with PBS (200 mg/L KCl, 200 mg/L KH2PO4, 8000 mg/L NaCl, 2160 mg/L Na2HPO4, pH 7.3) and the layers of adipose and epidermal tissue were removed.

The musculature of the body was chopped into pieces no larger than 5 mm. Finely chopped muscle tissue was placed in a syringe and a mixing tube was attached. On the other side of the mixing tube, a syringe containing 10 ml of 0.2% collagenase type II, preheated at 28 °C, was inserted. They were gently mixed for 5 minutes until the foreign body sensation disappeared. Sufficiently mixed tissue was placed in a 50 ml conical tube. 10 ml of culture medium with FBS was added and gently pipetted. After centrifugation at 800 g for 5 minutes, the supernatant was removed and suspended with PBS. Cells were separated from large clumps by sequential filtering using 1000, 100, 70, and 40 um cell strainers. After centrifugation at 800 g for 5 minutes, the supernatant was removed.

Th cells were suspended in 10 mL of serum-free medium containing 3% penicillin/streptomycin, 3% gentamicin, 3% ZellShield, 1.5% neomycin, and 0.3% bacitracin, and then 90 mL was added to the 500 mL triangular flask. They were placed in a shaking incubator, rotated at 300 RPM, and cultured at 28 °C for 7 days.

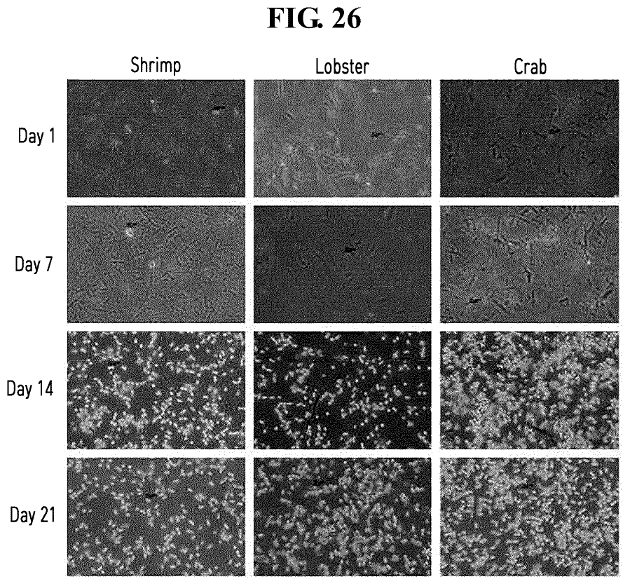

After 7 days, they were centrifuged at 800 g for 5 minutes, then the supernatant was removed, and the explant tissues were removed by 1 wash with PBS. After centrifugation at 800 g for 5 minutes, supernatant was removed, cells were suspended with 10 mL of serum-free medium containing 3% penicillin-streptomycin, 3% gentamicin, 3% ZellShield, 1.5% neomycin, and 0.3% bacitracin, and 90 mL was added into a 500 mL triangular flask and rotated at 300 RPM in a shaking incubator and cultured repeatedly at 28 °C. On the 14th day after cell culture, 1.5×10⁷ cells were transferred to a triangular flask containing 100 ml of a medium containing 0.3% bacitracin to dilute the remaining explant tissues and allow only the cells to proliferate. When the cell density reached 80 to 90%, the cells were sub-cultured at a 1:3 ratio. After repeated cultures, the cells were microscopically observed on days 1, 7, 14, and 21, and the results are shown in FIG. 26. As a result, the proliferation of cells with a size of 3 to 10um could be observed at 7 to 14 days after cell culture. As described above, cell lines were established after 3 weeks of sub-culturing. The cells were centrifuged at 800 g for 5 minutes, the supernatant was removed, and the cells were resuspended in 1 ml of Cellbanker 2 freezing medium to a concentration of 1×10⁹ cells/mL, and they were placed in a cryotube and stored at −80 °C for one day before being transferred to a liquid nitrogen tank.

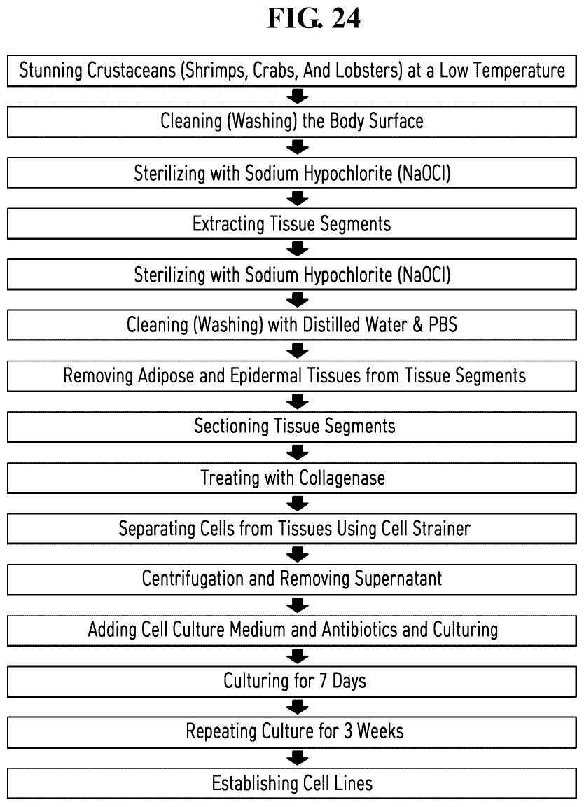

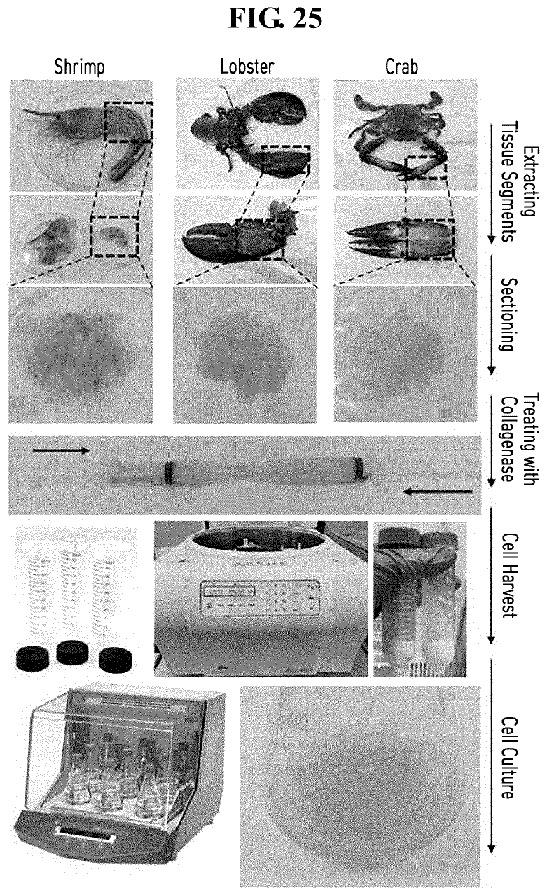

Schematic diagrams of the isolated cell and cell line establishment process described above are shown in FIGS. 24 and 25.

### Example 10. Separation of Cells From Crabs and Lobsters and Establishment of Cell Lines

Live crabs and lobsters were stunned by placing them on ice for 15 minutes before being sacrificed. The body segments were cleaned with a brush under running tap water and sterilized by soaking them in electrolytic sodium hypochlorite water (NaOCl) for 5 minutes three times. Each specimen's forelimbs were removed and the shells were removed. Muscle tissue was collected and sterilized by soaking it in electrolytic sodium hypochlorite water for 5 minutes, and then washed with sterile distilled water. Sections of the forelimb tissue were washed with PBS (200 mg/L KCl, 200 mg/L KH2PO4, 8000 mg/L NaCl, 2160 mg/L Na2HPO4, pH 7.3) and the muscle tissue was chopped into pieces no larger than 5 mm. Since the process of placing muscle tissue into a mixing tube and treating with 0.2% collagenase type II, the cell line was established using the same procedure as in Example 9.

### Example 11. Analysis of Cell Growth Under Culture Conditions

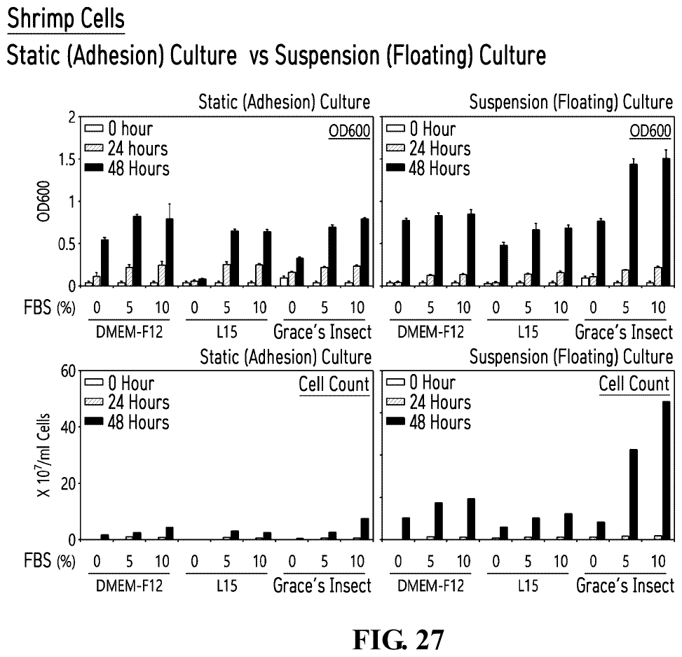

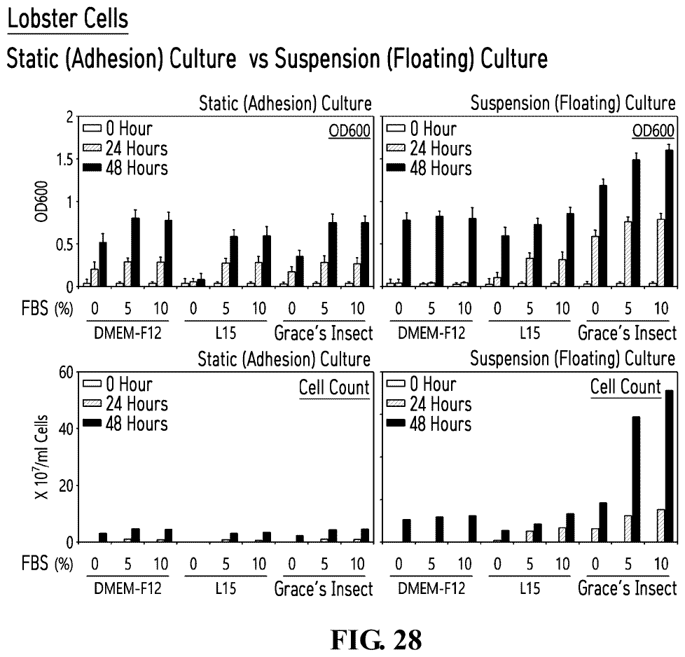

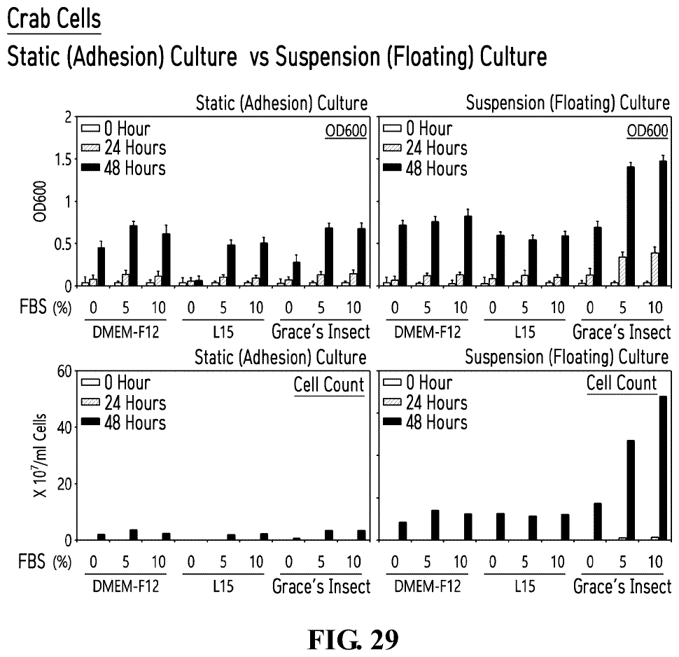

The method of isolating cells from previously known crustaceans has mainly been the static culture method. In order to find out whether there is a difference in cell growth according to the culture method, the cell proliferations were checked and compared in the case of suspension culture and static culture using the cell lines of shrimp, crab and lobster obtained from Examples 9 and 10. Specifically, the suspension culture was performed by placing cells in a 100 ml triangular flask and culturing them in a shaking incubator (at 28 °C at 250 rpm). Static culture was performed by placing cells in a T75 flask and incubating them in an incubator (28 °C). Using DMEM/F12, L15 or Grace's insect medium as a basic medium, the concentration of FBS was added at 0, 5, and 10% to confirm the cell growth. Cells were cultured at a cell density of 1.5×10⁵ cells/ml (10 ml in total). Optical density 600 nm (OD600) values were measured using absorption spectrometer at 0, 24, and 48 hours after cell culture, and cell counts were also confirmed directly using a hemocytometer. The results are shown in FIGS. 27 to 29.

As shown in FIG. 27 to 29, it could be found that the number of cells increased over time in the culture of shrimp, crab, and lobster cells up to 48 hours, using OD600 and cell counting results, and the proliferation rate of the cells increased as the concentration of FBS increased.

In the comparison of cell growth rates between suspension culture and static culture methods, the cell growth was higher in suspension culture than in static culture over culturing time. In particular, the number of cells was found to be the highest when suspension culture was performed under the condition of 10% FBS in Grace's insect medium, and the increase in the number of cells was more than 10 times higher than that of static culture under the same conditions.

From the above results, it was confirmed that differences in the growth rate of crustacean cells can also be caused by differences in the basic medium, and it was confirmed that crustacean cells had the highest growth rate in Grace's insect medium among the three basic media compared. In addition, it was confirmed that cells proliferate even in the absence of FBS, but the growth rate of cells increases when FBS is added. In addition, it was found that culturing cells using the suspension culture method can increase the growth rate of crustacean cells rather than the previously known static culture method.

### Example 12. Confirmation of Cell Line Establishment

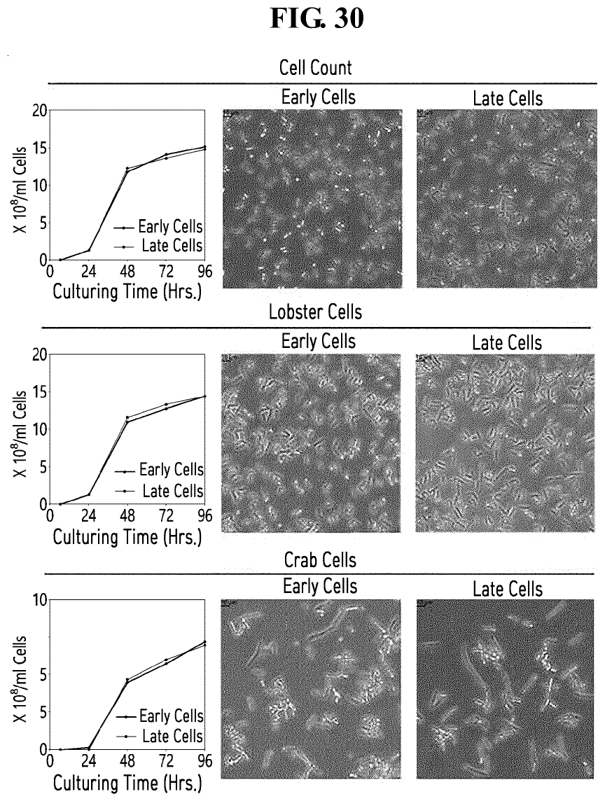

Separated cells from shrimp, crabs, and lobsters were checked to see if they were established as sustained growing cell lines. Specifically, the growth curves of cells (early cells) established through 3 weeks of sub-culturing of three species of cells separated from individuals, as in Examples 9 and 10, and cells (late cells) cultured for an additional month thereafter were confirmed. Cells with a cell density of 1.5×10⁵ cells/ml (10 ml in total) were inoculated into a 100 ml triangular flask and suspended in a shaking incubator (at 28 °C at 250 rpm) for 6, 24, 48, 72 and 96 hours. During culture, the number of cells was measured by counting the number of cells using a hemocytometer. The results are shown in FIG. 30.

As shown in FIG. 30, it was found that there was no difference in cell growth between early and late cells. In addition, as a result of observing the cells at different times, it was found that the cells had a similar shape.

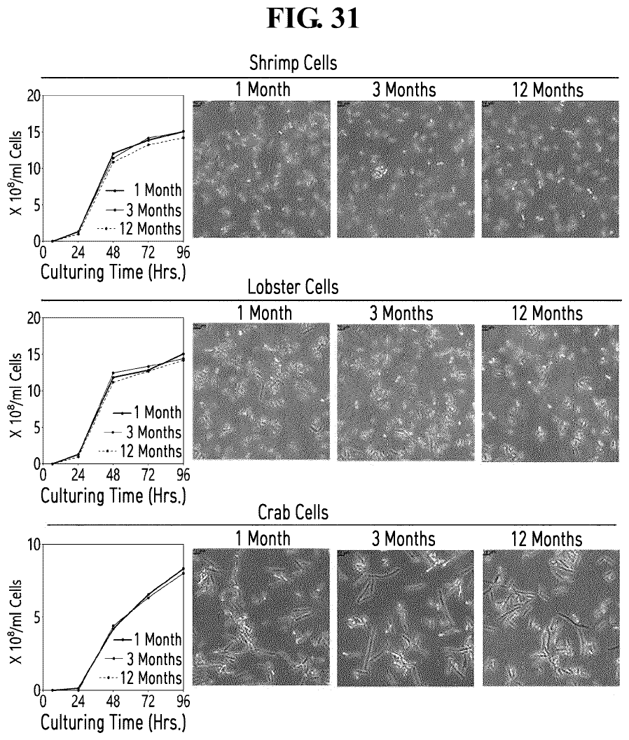

It was checked whether the established cell line maintained the growth even after long-term storage at ultra-low temperatures (−196 °C). Specifically, shrimp, crab, and lobster cells stored for 1, 3, and 12 months in liquid nitrogen were inoculated in a 100 ml triangular flask with a cell density of 1.5×10⁵ cells/ml (10 ml in total) and suspended in a shaking incubator (at 28 °C at 250 rpm) for 6, 24, 48, 72 and 96 hours. During culture, the number of cells was measured by counting the number of cells using a hemocytometer. The results are shown in FIG. 31.

As shown in FIG. 31, it was found that there was little difference in cell growth between cells stored for 1, 3 and 12 months. In addition, the cell shapes were found to be almost similar between groups.

From the above results, it was found that the crustacean cells of shrimp, crab, and lobster obtained in accordance with Examples 9 and 10 showed continuous growth without any change in their characteristics even after long-term culture, and that the cell growth was maintained even after long-term storage so that they were established as cell lines.

1. A medium composition for animal cell culture, comprising yeast extract or peptone.

2. A medium composition for animal cell culture, comprising a basic medium and yeast extract.

3. The medium composition according to claim 2, comprising the yeast extract or peptone in an amount of 0.01 to 10% (w/v).

4. The medium composition according to claim 2, wherein the animal cells are crustaceans cells.

5. The medium composition according to claim 2, wherein the animal cells are shrimp, crab, or lobster-derived cells.

6. The medium composition according to claim 2, wherein the medium is a serum-free medium.

7. The medium composition according to claim 2, wherein the culture is suspension culture.

8. The medium composition according to claim 2, wherein the basic medium does not include one or more species of vitamins selected from a group consisting of biotin, D-calcium pantothenate (D-Ca pantothenate), alpha-ketoglutaric acid (a-ketoglutaric acid), choline chloride, folic acid, i-inositol, niacin (nicotinic acid), pyridoxine-HCl, riboflavin, thiamine-HCl, and penta-aminobenzoic acid.

9. The medium composition according to claim 2, wherein the basic medium does not include vitamins.

10. The medium composition according to claim 2, wherein the basic medium does not include one or more species of organic acids selected from a group consisting of fumaric acid, malic acid, and succinic acid.

11. The medium composition according to claim 2, wherein the basic medium does not include organic acids.

12. A method of culturing animal cells, comprising culturing cells in a medium composition for animal cell culture comprising yeast extract and peptone.

13. A method of culturing animal cells, comprising culturing cells in a medium composition for animal cell culture comprising a basic medium and yeast extract.

14. A method of preparing a cell line, comprising separating a cell from a non-human animal; and
suspension-culturing the separated cell.

15. The method according to claim 14, wherein the non-human animal is a cow, sheep, pig, poultry, crustacean, or fish.

16. The method according to claim 15, wherein the crustacean is shrimp, crap, or lobster.

17. The method according to claim 14, wherein the suspension culture is performed for 1 to 42 days.

18. A cell line prepared by the method of preparing a cell line, comprising separating a cell from a non-human animal; and suspension-culturing the separated cell.

19. The method according to claim 14, further comprising performing a suspension culture for proliferating the cell line.

20. Cultured meat comprising a cell line prepared according to a method of preparing a cell line, comprising separating a cell from a non-human animal; and suspension-culturing the separated cell.

21. The medium composition according to claim 1, comprising the yeast extract or peptone in an amount of 0.01 to 10% (w/v).

22. The medium composition according to claim 1, wherein the animal cells are crustaceans cells.

23. The medium composition according to claim 1, wherein the animal cells are shrimp, crab, or lobster-derived cells.

24. The medium composition according to claim 1, wherein the medium is a serum-free medium.

25. The medium composition according to claim 1, wherein the culture is suspension culture.
